(19) United States
(12) Patent Application Publication
(10) Pub. No.: US 2018/0373700 A1
(43) Pub. Date: Dec. 27, 2018

(54) READER-DRIVEN PARAPHRASING OF ELECTRONIC CLINICAL FREE TEXT

(71) Applicant: KONINKLIJKE PHILIPS N.V., Eindhoven (NL)

(72) Inventors: Oladimeji Feyisetan FARRI, Yorktown Heights, NY (US); Sheikh Sadid Al HASAN, Cambridge, MA (US); Junyi LIU, Windham, NH (US)

(21) Appl. No.: 15/775,072

(22) PCT Filed: Nov. 21, 2016
(86) PCT No.: PCT/EP2016/078240

§ 371 Date: May 10, 2018

Related U.S. Application Data
(60) Provisional application No. 62/259,946, filed on Nov. 25, 2015.

(51) Int. Cl. G06F 17/27; G06F 17/24; G16H 70/20

(52) U.S. Cl. CPC G06F 17/2785; G06F 17/2795; G06F 17/2735; G16H 70/20; G06F 17/241

(57) ABSTRACT

A system (100) for understanding free text in clinical documents includes an information extraction engine (124) and a paraphrasing unit (140). The information extraction engine (124) extracts a selected sentence (118) from a clinical document (112) in response to an input. The paraphrasing unit (140) paraphrases the extracted sentence using a statistical machine translation model (142) trained using phrase sentence-alignment pairs (212) and outputs a constructed paraphrased sentence (320, 330, 410, 420, 430).

### FIELD OF THE INVENTION

The following generally relates to patient access of health records and natural language processing with specific application to patient review of electronically accessed clinical reports.

### BACKGROUND OF THE INVENTION

Patients are increasingly directly accessing electronic clinical reports, which are typically generated for a healthcare professional about the patient by another healthcare professional. The clinical report, such as a laboratory result, a diagnostic imaging result, physical examination, and the like, typically include free text. The free text includes medical terms, abbreviations, and jargon, which can be unintelligible to or difficult to understand by the patient. The free text is unstructured text in sentence form. This access can in part be driven by patient desire to understand and participate in their healthcare decisions. The access can in part be driven by the use of multiple healthcare providers who maintain separate healthcare records and a need to understand a holistic view of health information from different providers or sources.

In trying to make sense of the free text in the clinical documents, patients typically use internet search engines to look up medical terms. The search results include definitions and large quantities of documents, which do not consider the context of the medical term in the sentence of the clinical report. Some approaches to demystifying the free text include natural language processing techniques that identify medical terms and map the terms to an ontology. Mapping the terms to an ontology standardizes the terms, but the sentence context is absent and the nomenclature remains based on understanding by the healthcare professional and not the patient.

### SUMMARY OF THE INVENTION

Aspects described herein address the above-referenced problems and others.

The following describes a method and system for displaying sentences as paraphrased sentences selected from free text in a clinical document. A statistical machine translation model is trained with phrase sentence-alignment pairs to paraphrase an indicated sentence in a patient clinical document. Phrase sentence-alignment pairs can include textual entailment. Phrase sentence-alignment pairs are constructed from an annotated corpus of clinical reports. Paraphrasing can include emoticons. Paraphrasing can include reader feedback, which further or alternatively paraphrases a sentence. Paraphrasing can include extensions of the statistical machine translation model encounter of new terms with paraphrasing based on mapped ontological concepts and/or synonyms.

In one aspect, a system for understanding free text in clinical documents includes an information extraction engine and a paraphrasing unit. The information extraction engine extracts a selected sentence from a clinical document in response to an input. The paraphrasing unit paraphrases the extracted sentence using a statistical machine translation model trained using phrase sentence-alignment pairs and outputs a constructed paraphrased sentence.

In another aspect, a method of understanding free text in clinical documents includes in response to an input, extracting a selected sentence from a clinical document. The extracted sentence is paraphrased using a statistical machine translation model trained using phrase sentence-alignment pairs, which outputs a paraphrased sentence.

In another aspect, a system for understanding free text in clinical documents includes an information extraction engine and a paraphrasing unit. The information extraction engine, in response to an input, extracts a selected sentence from a clinical document. The paraphrasing unit paraphrases the extracted sentence using a statistical machine translation model trained using phrase sentence-alignment pairs obtained from an annotated corpus of clinical reports clustered by tuples which include a diagnosis, a test, and a treatment, and displays the paraphrased sentence on a display device.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 schematically illustrates an embodiment of a reader-driven paraphrasing of electronic clinical free text system.

FIG. 2 schematically illustrates an embodiment of a reader-driven paraphrasing algorithm.

FIG. 3 illustrates exemplary paraphrases of a selected sentence.

FIG. 4 illustrates exemplary paraphrases of another selected sentence.

### DETAILED DESCRIPTION OF EMBODIMENTS

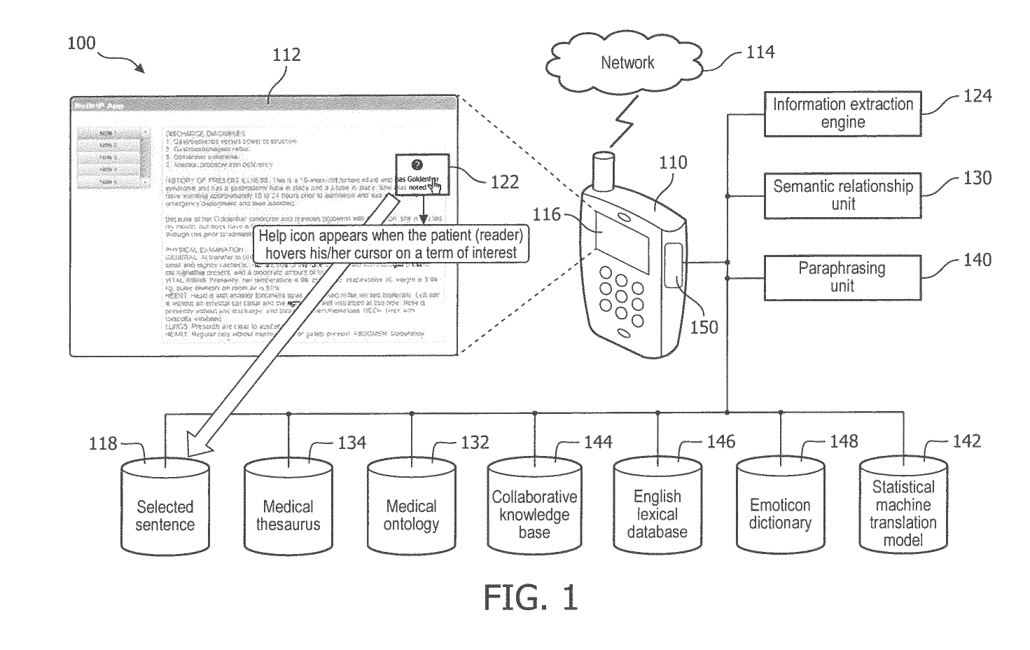

Initially referring to FIG. 1, an embodiment of a reader-driven paraphrasing of electronic clinical free text system 100 is schematically illustrated. A computing device 110, such as a smartphone, laptop computer, desktop computer, tablet, body worn device, and the like, is configured to access a clinical document 112 with free text. The access can be local or remote. For example, the clinical document 112 can be retrieved from local memory of the computing device 110 or retrieve through a web portal, cloud storage, and the like using a network 114, such as the Internet.

The clinical document 112 is displayed on a display device 116 of the computing device 110. A sentence 118 is selected with an input device 120, such as a touch screen, microphone, mouse, keyboard and the like. For example, illustrated in FIG. 1 is a help box 122 that appears when a cursor hovers over a term in the sentence 118 or the sentence 118. An input, such as a tap on a touch screen or a mouse click selects the sentence 118. In another example, a voice input of “what is Goldenhar Syndrome?” can select a first sentence with the term “Goldenhar Syndrome.”

An information extraction engine 124 receives the input, and extracts the selected sentence 118 from the free text document. The input can be a physical position or location in the displayed clinical document 112 and/or a term or phrase used in the displayed clinical document 112. The extraction can include converting the format of the document, such as an image representation to character representation. The extraction includes tokenizing characters into words and sentence boundary detection. The extraction includes identification of phrases, such as noun phrases or predicate phrases. The information extraction engine 124 uses natural language processing (NLP) techniques to process the clinical document 112 with free text to identify the sentence and phrases within the selected sentence. An example of such techniques can be found in an application entitled “Algorithmic Design for Semantic Search and Extraction of Active Diagnoses from Clinical Documents” filed on Mar. 9, 2015 as application No. 62/130,141.

In one embodiment, the system 100 includes a semantic relationship unit 130 that can map terms, e.g. words and/or phrases, in the extracted sentence 118 to a medical ontology 132 and/or medical thesaurus 134. For example, using Systematized Nomenclature of Medicine-Clinical Terms (SNOMED CT) and/or Unified Medical Language System® (UMLS®) Metathesaurus®, terms are mapped to target concepts, e.g. concept ID of the Metathesaurus®. Other mappings can include ICD-10, Galen and the like. The mapping can include identifying negated concepts, or disambiguating acronyms and/or abbreviations based on sentence context. In one embodiment, the sentence context can include the document context and/or context of a portion of the document, such as a header information. Examples of such techniques can be found in the application entitled “Algorithmic Design for Semantic Search and Extraction of Active Diagnoses from Clinical Documents.”

A paraphrasing unit 140 paraphrases the extracted sentence 118 using a trained statistical machine translation model 142. The paraphrased sentence can replace the selected sentence 118 in the displayed selected sentence 118 or can be provided separately, such as a pop-up box, bubble, screen, audio output and the like. The trained statistical machine translation model 142 is trained using phrase sentence-alignment pairs constructed from an annotated corpus of clinical reports. The phrases are phrases which are translated as sentence-alignments. The trained statistical machine translation model 142 can include inference rules and/or templates, e.g. hybrid machine translation model. The trained statistical machine translation model 142 can include training with collaborative knowledge bases 144, such as Freebase, Wikipedia, and the like. The trained statistical machine translation model 142 can include training with English lexical databases 146, such as WordNet. For example, descriptions and word senses, i.e. word glosses, from definitions in WordNet can be used for training. In one embodiment, training can include an emoticon dictionary 148. The translation can include substitution of words/phrases. The translation can include sentence reorganization. The translation can include compression, e.g. fewer words and/or simplification, e.g. fewer different words. In one embodiment, the translation can include textual entailment, in which replacement text entails the meaning of the original sentence based on a hypothesis, e.g. unidirectional translation.

In some instances, training with the collaborative knowledge bases 144, English lexical databases 146 and/or an emoticon dictionary 148 orients nomenclature of the paraphrased sentence to that of a patient. In some instances, training which uses text entailment orients the nomenclature of the paraphrased sentence to that of a patient. In some instances, use of mapped concepts with the semantic relationship unit 130 allow extension of the trained statistical machine translation model 142 to extend translation of new encountered terms, which are mapped using the medical thesaurus 134 and/or medical ontology 132 to phrases within the scope of the training based on the mapped target concept. For example, a first term is mapped to concept A and the first term, e.g. a noun phrase, is used to train the statistical machine translation model 142. A second term is encountered by the statistical machine translation model 142, and is new. The second term is mapped to the concept A by the semantic relationship unit 130, and the statistical machine translation model 142 translates the sentence with the second term based on the mapping to concept A and/or in turn to the first term.

The information extraction engine 124, the semantic relationship unit 130, and the paraphrasing unit 140 comprise one or more configured processors 150, e.g., a microprocessor, a central processing unit, a digital processor, and the like) are configured to execute at least one computer readable instruction stored in a computer readable storage medium, which excludes transitory medium and includes physical memory and/or other non-transitory medium. The processor 150 may also execute one or more computer readable instructions carried by a carrier wave, a signal or other transitory medium. The processor 150 can include local memory and/or distributed memory. The processor 150 can include hardware/software for wired and/or wireless communications. For example, the lines indicate communications paths between the various components which can be wired or wireless. The processor 150 can comprise the computing device 110, such as a desktop computer, a server, a laptop, a mobile device, a body worn device, distributed devices, combinations and the like.

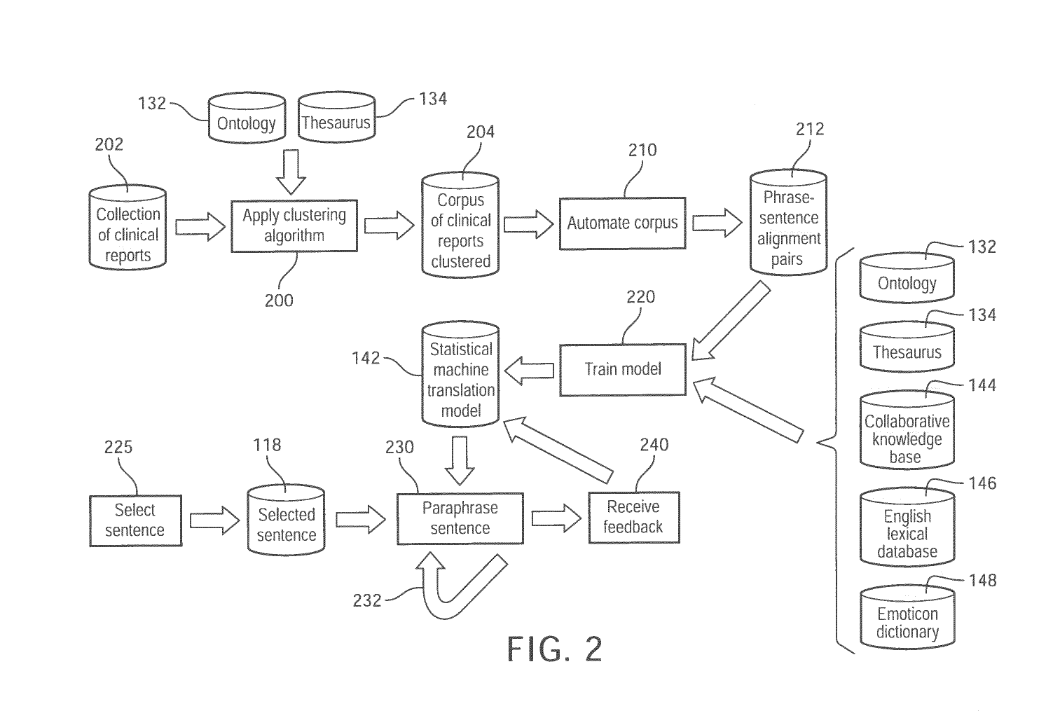

With reference to FIG. 2, an embodiment of a reader-driven paraphrasing algorithm is schematically illustrated. At 200, a clustering algorithm is applied to a collection of clinical reports 202, which creates a corpus of clustered clinical reports 204 by tuples of (diagnosis, test, treatment). The collection of clinical reports 202 includes free text sentences. For example, the collection of clinical reports 202 can be obtained from electronic medical records (EMR), departmental clinical reports, and the like, with personal identification information removed. The clustering algorithm can include conversion of formats of reports, such as image representation to character representation. The clustering algorithm can include tokenization of words. The clustering algorithm can include mapping of terms to the ontology 132 and/or the thesaurus 134 to obtain consistent tuples of diagnosis, test and treatment. For example, all clinical reports with (acute respiratory distress syndrome (ARDS), chest x-ray, and mechanical ventilation) belong to the same cluster. The chest x-ray test can include semantic equivalents, such as chest computed tomography (CT), thorax CT, and the like. Each cluster represents a large comparable corpus with phrases and sentences likely similar in meaning.

At 210, the corpus of clustered clinical reports 204 is annotated, which identifies phrase sentence-alignment pairs 212. The identification can be using phase-alignment models known in the art or manually using clinical domain experts. In one embodiment, the identified phrase sentence-alignment pairs 212 can include a mapping to the target concept based on the ontology 132 and/or thesaurus 134.

At 220, the statistical machine translation model 142 is trained using the phrase sentence-alignment pairs 212. In some instances, the phrase sentence-alignment pairs 212 include the context of the sentence in the training, e.g. the relationship between the words used in the sentence. The training can include other corpus, such as descriptions and examples from the ontology 132, the thesaurus 134, the collaborative knowledge bases 144, the English lexical database 146 or the emoticon dictionary 148. The training can include bootstrapping, which balances the weighting of inference rules and/or templates during initial training. The inference rules direct the probabilistic replacement text with paraphrasing. Templates can be used to direct different sets of inference rules. For example, templates can be used to direct inference rules to readers with different characteristics or preferences, such as use of emoticons or weighting on one of more corpus during the training.

At 225, a sentence (118) is selected and extracted from the free text of a displayed clinical document (112) in response to an input. The input can include a spatial location indicative of the sentence (118) or a word in the sentence (118). The input can include a word or term from the sentence (118).

At 230, the trained statistical machine translation model 142 paraphrases the extracted sentence 118. The paraphrased sentence is output, e.g. displayed. The paraphrased sentence can be displayed as an overlay of the selected sentence, e.g. replaces the sentence, or separately, such as a separate box, bubble display, audio output, etc. The paraphrasing can include translation, e.g. bidirectional. The paraphrasing can include textual entailment, e.g. unidirectional. In some instances, textual entailment addresses redundancy and ensures conciseness with accuracy. Textual entailment includes creating vector space representations of the selected sentence/partially paraphrased sentence and recognizes if a sentence in a pair of sentences or conjunctive clauses has a textual entailment in either direction. The paraphrasing can include emoticons. The paraphrasing can include sentence reorganization, e.g. different ordering of words, different ordering of noun or predicate, and the like. The paraphrasing can include compression/simplification, e.g. fewer words. The paraphrasing can include different words and/or phrases, e.g. synonyms, synonyms based on original word, semantically equivalent words or visual representations based on target concept, entailment, combinations and the like.

The paraphrasing can include re-paraphrasing 232. For example, an extracted sentence is paraphrased. Another input, such as another screen tap or mouse click indicates that the paraphrasing is still not understandable, and the statistical machine translation model 142 replaces the first paraphrased sentence with a second paraphrased sentence. The inputs can include user specific preferences such as using emoticons or another template as additional input to the model in selecting the next paraphrasing.

At 240, the statistical machine translation model 142 can receive feedback. The feedback can include acceptance or non-acceptance of the paraphrasing. The feedback can include feedback from a plurality of computing devices 110. The feedback can include a rating of the paraphrasing, e.g. scale indicator such as number of stars. The feedback can be used by the statistical machine translation model 142 to adapt paraphrasing statistically according to accepted and/or non-accepted paraphrases, e.g. adjust weightings and/or adjust inference rules.

The above may be implemented by way of computer readable instructions, encoded or embedded on computer readable storage medium, which, when executed by a computer processor(s), cause the processor(s) to carry out the described acts. Additionally or alternatively, at least one of the computer readable instructions is carried by a signal, carrier wave or other transitory medium.

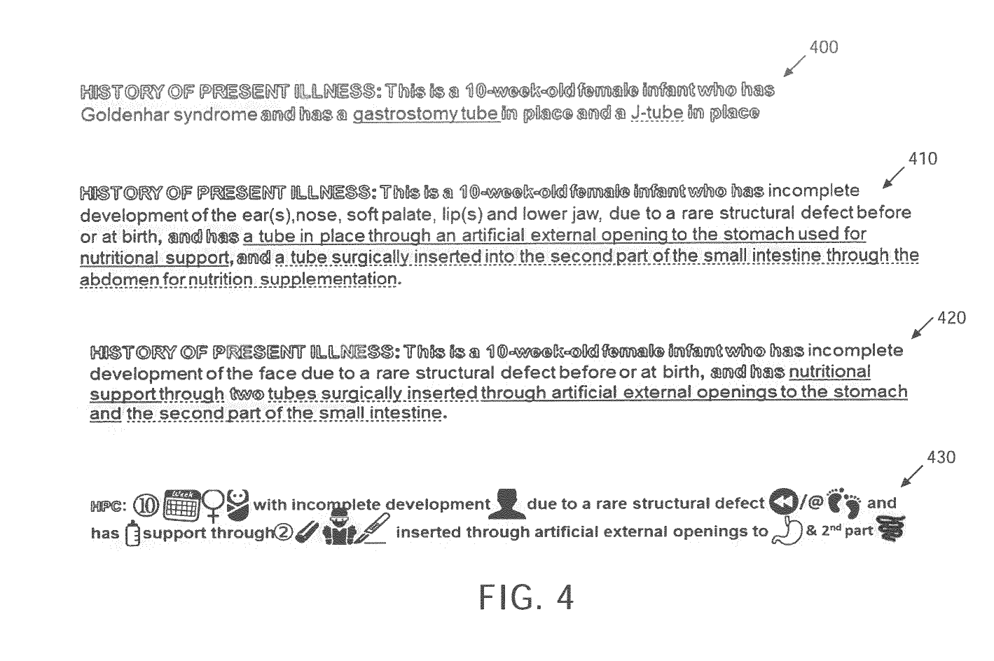

With reference to FIG. 3, exemplary paraphrases of an extracted sentence 300 are illustrated. The extracted sentence of “Atherosclerotic plaques can be associated with acute and chronic diseases” includes a phrase “atherosclerotic plaques” and a phrase “acute and chronic diseases.”

The phrases in context can be entailed with a hypothesis sentence 310 “Acute myocardial infarction (AMI) typically results from a ruptured atherosclerotic plaque which triggers thrombus formation, total occlusion of a coronary artery and necrosis of the cardiac muscle cells.” The concept of “acute and chronic diseases” is represented in the hypothesis as AMI, thrombus formation, and necrosis of the cardiac muscle cells in the context of the plaque. The atherosclerotic plaque is represented in the context of acute and chronic diseases with ruptured atherosclerotic plaque, total occlusion of a coronary artery. The sentence is reorganized. The sentence includes an acute disease of AMI related to the atherosclerotic plaque first, the ruptured atherosclerotic plaque second, and the chronic diseases including thrombus formation, total occlusion of a coronary artery and necrosis of the cardiac muscle cells third.

The hypothesis sentence 310 is paraphrased in 320 as “A heart attack usually occurs when there is a break in an abnormal collection of cholesterol and fibrous tissue within the wall of a blood vessel, initiating the formation of blood clots, complete blockage of a blood vessel in the heart and premature death of heart muscles cells.” The paraphrasing replaces medical based phrases with phrases in a nomenclature oriented toward the collaborative knowledge bases 144 and/or the English lexical database 146 used to train the statistical machine translation model 142. For example, “ruptured” is replaced with a synonym “break.” “Atherosclerotic plaque” is replaced with “an abnormal collection of cholesterol and fibrous tissue within the wall of a blood vessel.” “Triggers” is replaced with “initiating” and “thrombus formation” is replaced with “blood clots.” “Total occlusion of a coronary artery” is replaced with “complete blockage of a blood vessel in the heart.” “Necrosis of cardiac muscle cells” is replaced with “premature death of heart muscle cells.”

The extracted sentence 300 is alternatively paraphrased using emoticon paraphrasing 330. The emoticon paraphrased sentence 330 is “A [symbol] usually occurs when [symbol] [symbol] , initiating [symbol] , complete [symbol] & premature [symbol] .” The emoticons include pictorial representations of phrases. For example, “heart attack” is represented as [symbol] .

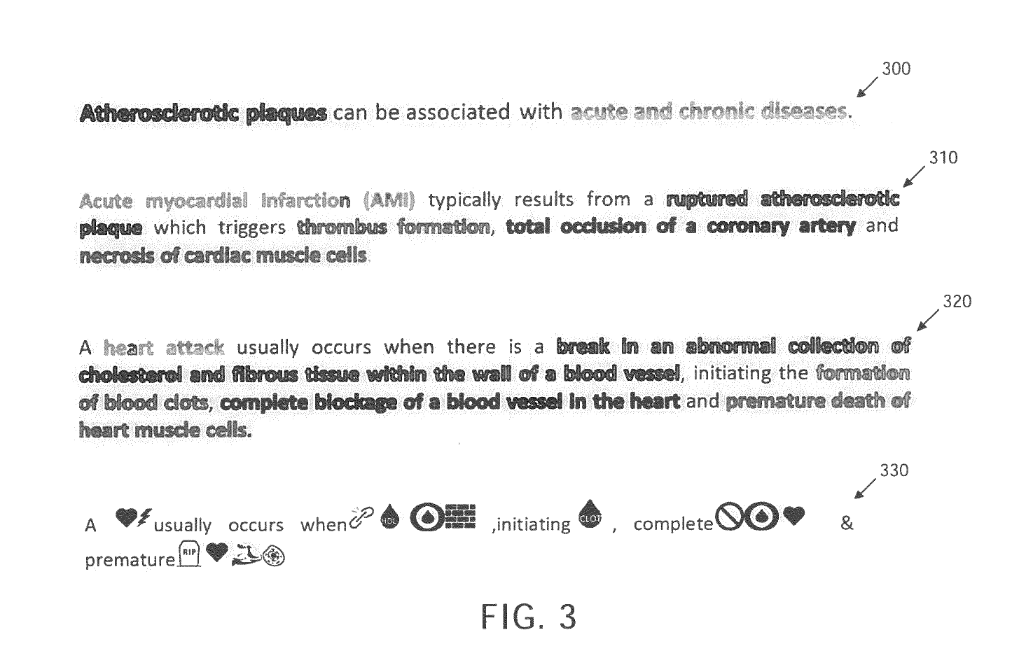

With reference to FIG. 4, exemplary paraphrases of another extracted sentence 400 are illustrated. The extracted sentence 400 is “History of Present Illness: This is a 10-week-old female infant who has Goldenhar syndrome and has a gastrostomy tube in place and a J-tube in place.” A paraphrased sentence 410 replaces in context the phrases “Goldenhar syndrome,” “gastrostomy tube in place” and “J-tube in place.” The paraphrased sentence 410 is “History of Present Illness: This is a 10-week-old female infant who has incomplete development of the ear(s), nose, soft palate, lip(s) and lower jaw, due to a rare structural defect before or a birth, and has a tube in place through an artificial external opening to the stomach used for nutritional support, and a tube surgically inserted into the second part of the small intestine through the abdomen for nutritional supplementation.”

In the paraphrased sentence 420, textual entailment is used to reduce the sentence length. “Of the ear(s), nose, soft palate, lip(s) and lower jaw” is entailed within “of the face.” This phrase translation is unidirectional. “A tube in place through an artificial external opening to the stomach used for nutritional support, and a tube surgically inserted into the second part of the small intestine through the abdomen for nutritional supplementation” is entailed with “nutritional support through two tubes surgically inserted through artificial external openings to the stomach and the second part of the small intestine.” The second entailment includes the recognition of the conjunctive clauses. In the paraphrased sentence 430, emoticons are used to represent phrases, e.g. one or more words.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A system for paraphrasing free text in clinical documents, comprising:
an information extraction engine is configured to extract a selected sentence from a clinical document in response to an input; and
a paraphrasing unit configured to paraphrase the extracted sentence using a statistical machine translation model trained using phrase sentence-alignment pairs constructed from a corpus of clinical documents and output a constructed paraphrased sentence, wherein phrase sentence-alignment pairs comprise a phrase in a context of a sentence aligned and paired with another phrase in a context of another sentence.

2. The system according to claim 1, wherein paraphrasing includes textual entailment with a meaning of the extracted sentence entailed within the paraphrased sentence using different words.

3. The system according to claim 1, wherein the corpus of clinical documents includes documents with free text sentences.

4. The system according to claim 3, wherein the corpus of clinical documents includes an annotated corpus of clinical documents with free text clustered by tuples which include a diagnosis, a test, and a treatment.

5. The system according to claim 1, wherein the statistical machine translation model is trained with at least one of a collaborative knowledge base, an English Lexical database or an emoticon dictionary.

6. The system according to claim 1, wherein the paraphrasing unit is further configured to:
in response to a second input, re-paraphrase the extracted sentence using the statistical machine translation model, which uses an alternative translation.

7. The system according to claim 1, wherein the paraphrasing unit is further configured to:
receive feedback of acceptance of the paraphrasing and modify at least one of an inference rule or a weight used by the statistical machine translation model.

8. The system according to claim 1, wherein the paraphrased sentence is different from the extracted sentence in at least one of sentence reorganization, compression, or simplification.

9. The system according to claim 1, wherein the paraphrased sentence includes emoticons.

10. The system according to claim 1, further including:
a semantic relationship unit configured to map terms in the extracted sentence to a target concept based on at least one of a medical ontology or a medical thesaurus;
wherein the paraphrasing unit in response to encountering a new term in the extracted sentence uses the mapped target concept to paraphrase the new term.

11. A method of paraphrasing free text in clinical documents, comprising:
in response to an input, extracting a selected sentence from a clinical document; and
paraphrasing the extracted sentence using a statistical machine translation model trained using phrase sentence-alignment pairs constructed from a corpus of clinical documents, which outputs a paraphrased sentence, wherein phrase sentence-alignment pairs comprise a phrase in a context of a sentence aligned and paired with another phrase in a context of another sentence.

12. The method according to claim 11, wherein paraphrasing includes:
textually entailing a meaning of the selected sentence within the paraphrased sentence in a unidirectional translation.

13. The method according to claim 11, further including:
applying a clustering algorithm to a corpus of clinical documents with free text sentences by tuples which include a diagnosis, a test, and a treatment;
annotating the clustered corpus of clinical documents to obtain phrase sentence-alignment pairs; and
training the statistical machine translation model using the phrase sentence-alignment pairs.

14. The method according to claim 13, wherein training includes training with at least one of a collaborative knowledge base, an English Lexical database or an emoticon dictionary.

15. (canceled)

16. (canceled)

17. (canceled)

18. (canceled)

19. (canceled)

20. (canceled)

21. A computer readable storage medium comprising instructions for paraphrasing first text in clinical documents, which when executed cause a processor to:
in response to an input, extract a selected sentence from a clinical document; and
paraphrase the extracted sentence using a statistical machine translation model trained using phrase sentence-alignment pairs constructed from a corpus of clinical documents, which outputs a paraphrased sentence, wherein phrase sentence-alignment pairs comprise a phrase in a context of a sentence aligned and paired with another phrase in a context of another sentence.
